(19) United States
(12) Patent Application Publication
(10) Pub. No.: US 2023/0201016 A1
(43) Pub. Date: Jun. 29, 2023

(54) MODULAR WEARABLE PAD(S) FOR THERAPEUTIC COCCYX REALIGNMENT

(71) Applicants: Joseph R. O'Brien, McLean, VA (US); Babak Arvanaghi, McLean, VA (US)

(72) Inventors: Joseph R. O'Brien, McLean, VA (US); Babak Arvanaghi, McLean, VA (US)

(21) Appl. No.: 18/081,577

(22) Filed: Dec. 14, 2022

Related U.S. Application Data
(60) Provisional application No. 63/293,138, filed on Dec. 23, 2021.

(51) Int. Cl. A61F 5/00

(52) U.S. Cl. CPC A61F 5/0093

(57) ABSTRACT

A wearable undergarment with removable pads designed in a modular configuration customized to the user by an attending physician. Undergarment provides gradual mechanical realignment and pain relief for injury to the coccyx resulting in coccydynia. Customized modular pads provide the user with variable compression, particularly underneath the buttocks and surrounding region while sitting.

This application claims the benefit of U.S. Provisional Application No. 63/293,138, filed Dec. 23, 2021, which is incorporated herein by reference in its entirety.

### TECHNICAL FIELD

Embodiments of the present invention generally relate to devices for relieving back pain. In particular, the present invention pertains to wearable pads that mechanically realign the coccyx and surrounding areas.

### BACKGROUND

The coccyx, also referred to as the tailbone, is a small triangular bone at the bottom of the spinal column. Bruising or fracturing the coccyx and injury or strain to the surrounding muscles and ligaments can lead to localized pain in the tailbone region called coccydynia. Common causes of coccydynia include falling, repetitive strain, poor posture, pregnancy and childbirth, and obesity. The pain worsens in the sitting position since the coccyx and the bottom of the pelvis bear most of the weight when in this position. While coccydynia can resolve on its own with or without moderate treatment, it can become chronic and debilitating for some individuals.

The current outpatient treatments for coccydynia include a coccygeal nerve block, massage therapy, stretching and posture exercises, acupuncture, and transcutaneous electrical nerve stimulation. These treatments may require physician supervision and do not always result in extended pain relief. In addition, the existing targeted pain management options focus primarily on pressure relief.

### BRIEF DESCRIPTION OF THE FIGURE

FIG. 1 illustrates a garment 100, according to an embodiment.

Further embodiments, features, and advantages of the present invention, as well as the operation of the various embodiments of the present invention, are described below with reference to the accompanying figure.

### DESCRIPTION OF EMBODIMENTS

This invention introduces an alternative treatment option using customized modular wearable pads that mechanically realign the user's lower back to alleviate pain caused by coccydynia. The therapeutic modular pads are tailored specifically to the individual by an attending physician and are adjusted accordingly.

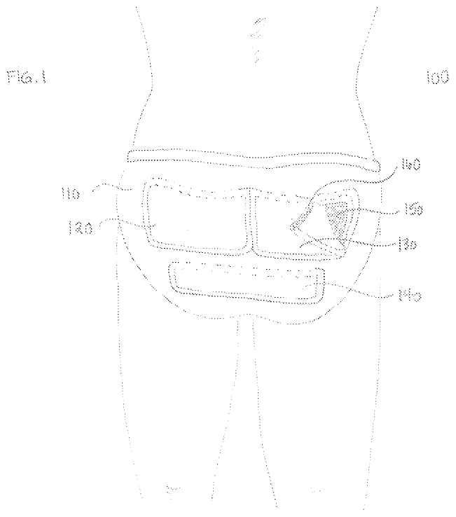

Referring to the embodiment of FIG. 1, the apparatus 100 provides the user with a wearable garment or undergarment with one or more attached modular pockets wherein a removable pad 150 may be inserted for the purpose of mechanical realignment of the coccyx and surrounding region of the user over time. According to this embodiment, the apparatus 100 is shown with a first modular pocket 120, a second modular pocket 130, and a third modular pocket 140 strategically designed by a physician or other caregiver to accommodate the user's medical treatment plan. In various embodiments, the quantity, shape, and size of the modular pockets are customizable by the physician to provide variable compression underneath the buttocks and upper leg region when sitting.

In FIG. 1, this embodiment shows the removable pad 150 inserted through an opening in the top side of the second modular pocket 130. The removable pad 150 remains inside the modular pocket 130 by a hook and loop closure 160 at the top side of the pocket. In the absence of a pocket, another embodiment may attach the removable pad 150 to the user's own garment by a removable adhesive or other attachment mechanism. In the embodiment shown, the removable pad 150 consists of a gel material inside a packet, where the resulting pad has a physician-prescribed thickness. The gel material in another embodiment may be injectable. The thickness of the removable pad 150 may range from 5 mm to 2 cm with the gel, depending on its location and the needs of the patient. The user's physician may determine the appropriate thickness based on variables such as medical scans, degree of pain, and posture for example. In an embodiment, the prescribed thickness may be achieved by using more than one pad, each having a predetermined thickness, in a stacked configuration.

The apparatus 100 is customized by the user's physician according to results from observation and medical scans. The user's physician determines the duration for which the apparatus 100 is to be worn through medical prescription, but the apparatus 100 may be freely removable by the user.

While specific configurations and arrangements are discussed above, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other systems and applications.

What is claimed is:

1. A wearable garment, comprising:
at least one pocket situated to be beneath a patient's buttocks or upper leg region when the patient is sitting; and
at least one removeable pad inserted through an opening in the at least one pocket.

2. The garment of claim 1, wherein the quantity, shape, and size of the at least one removeable pad is prescribed by a physician to provide a prescribed degree of lift underneath the buttocks or upper leg region when the patient is seated.

3. The garment of claim 1, wherein the at least one removeable pad is attached inside the pocket by a removeable adhesive or a hook and loop attachment means.

4. The garment of claim 1, wherein the at least one removeable pad comprises a gel material.

5. The garment of claim 1, wherein the at least one removeable pad has a thickness from 5 mm to 20 mm.

6. The garment of claim 2, wherein the thickness of the at least one removeable pad is determined by the physician based on variables comprising one or more of medical scans, degree of pain, and posture of the patient.

7. The garment of claim 1, wherein the garment is an undergarment.

8. The garment of claim 1, wherein a thickness of the one or more removeable pad is prescribed by a physician.

9. The garment of claim 4 wherein the gel material is injectable into the one or more removeable pad.
